(19) United States
(12) Patent Application Publication
(10) Pub. No.: US 2007/0141673 A1
(43) Pub. Date: Jun. 21, 2007

(54) Tool for quantitative real-time analysis of viral gene expression dynamics in single living cells

(76) Inventors: Asier Saez-Cirion, Massy (FR); Marie-Anne Nicola, Clamart (FR); Gianfranco Pancino, Paris (FR); Spencer Shorte, St. Piat (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/602,487

(22) Filed: Nov. 21, 2006

Related U.S. Application Data
(60) Provisional application No. 60/738,205, filed on Nov. 21, 2005.

(51) Int. Cl. C12Q 1/68; C12P 21/06

(52) U.S. Cl. 435/69.1; 435/6

(57) ABSTRACT

The invention provides a method allowing the detection and the quantitative real-time measurement, at the single living cell level, of viral replication using a bioluminescent reporter gene and a digital light detection device sensitive to detect single photons with high efficiency and assign them a lateral x,y coordinate and precise temporal incidence (i.e. a “time point”), wherein the spatial and temporal characteristics of bioluminescence is indicative at the single living cell level of viral replication.

### CROSS-REFERENCE TO RELATED APPLICATION

This application is based on and claims the benefit of U.S. Provisional Application Ser. No. 60/738,205, filed Nov. 21, 2005, (Attorney Docket No. 03495.6113). The entire disclosure of this Provisional application is relied upon and incorporated by reference herein.

### FIELD OF THE INVENTION

The methods and kits of the invention provide new tools for the quantitative real-time measurement at the single living cell level of viral replication. According to the invention, the term “viral” indifferently refers to DNA and RNA viruses.

### BACKGROUND OF THE INVENTION

Studies on the regulation of viral replication upon infection of the target cells have provided important information on the viral and host factors that influence pathogenesis.

Fluorescent viral protein conjugates have been used to study HIV infection in single cell models (Mc Donald et al. (2002) J. Cell Biol. 159:441-452). However, these labelling strategies do not support visualisation of virus at, or beyond, the point of pre-integration complex formation. Furthermore, fluorescence based methods for following HIV gene expression lack sensitivity and duration of recording.

Viral vectors, for example HIV vectors, bearing the luciferase reporter gene have been widely used to quantify single cycle viral replication in target host cells, constituting a priceless tool in the expansion of the knowledge on the interaction of the virus with host cells (Chen et al. (1994) J. Virol. 68:654-603; Connor et al. (1995) Virology 206:935-44; Liu et al. (1996) Cell 86:367-77, Mariani et al. (2003) Cell 114:21-31).

However, to date, these studies have been limited to steady-state (end-point) measurement of viral infection levels, based upon luminometer assay of chemiluminescence (bioluminescence) light production detected in cell lysate suspensions. So, despite their usefulness, such methods fail to provide real-time read-out on the dynamics of infection, and lack single-cell resolution neglecting to take into account functional differences arising from cell-cell heterogeneity.

It is therefore of great interest that bioluminescence is to date now finding itself a choice methodology for real-time analysis of long-term changes in gene expression (and other physiological parameters) at the whole animal, tissue and even at the single cell level (Doyle et al. (2004) Cell Microbiol. 6:303-176; Greer et al. (2002) Luminescence 17:43-74; Rutter et al. (1995) Curr. Biol. 5:890-9; Shorte et al. (2002) Endocrinology 143:1126-33).

Chemiluminescent reporter genes are based upon the use of naturally occurring enzymes that produce continuous visible light emission in the presence of specific natural substrate and in the absence of any excitation light (Alam et al. (2003) In S. C. Makrides (ed.), Gene transfer and expression in mammalian cells, vol. 38. Elsevier Science Publishing, The Netherlands), making phototoxic effects negligible. In the specific case of luciferase, the photo-protein emits light in the presence of its substrate D-luciferin, plus reaction cofactors, O₂ and ATP (Ignowski et al. (2004) Biotechnol. Bioeng. 86:827-34). Cell membranes are permeable to D-luciferin (thereby facilitating the delivery of this substrate into cells or tissues expressing the luciferase enzyme); and neither luciferase expression, nor the presence of its substrate luciferin are toxic for living tissues. Given non-limiting intracellular concentrations of the enzymatic reaction cofactors (O₂ and ATP) and substrate, the resulting bioluminescent signal is rendered quantitative inasmuch as the levels of gene expression may be extrapolated as a function of this signal (Rutter et al. (1998) Chem. Biol. 5:R285-90).

To date, in the field of viral pathology, the luciferase imaging technology has been limited to the real-time analysis of viral promoter directed gene expression in stable transfected single cells (White et al. (1995) J. Cell Science 108, 441-455). Although this approach is a useful tool to acquire a better understanding of the regulation of a viral gene transcription, it does not allow gaining information regarding the more complex phenomenon of viral replication.

There is a need for a method allowing to gain deeper knowledge on the extent to which single cell heterogeneity in cells, particularly in primary cells, can influence viral infection, particularly HIV infection, dynamics, and a greater need still to be able to monitor the kinetics of viral replication in situ.

The problem, which the invention proposes to solve, is to provide a method allowing the detection and the quantitative real-time measurement, at the single living cell level, of viral replication.

The method of the invention is based on the use of a bioluminescent reporter gene to follow viral replication inside single living cells.

The method is also based on a highly sensitive digital light detection imaging device equipped with an Imaging Photon Detector, which assignates an x,y-coordinate and time point for each detected photon.

According to the invention, the bioluminescent reporter gene is inserted in the viral genome. During viral replication, when the viral genome is transcribed into mRNA coding for viral proteins, the bioluminescent reporter gene is simultaneously transcribed. Then, the bioluminescent reporter gene transcripts are translated into the bioluminescent reporter protein. If bioluminescent reporter protein light emission is induced, the emitted light is proportional to the number of bioluminescent reporter gene copies and their transcription rate. As the number of bioluminescent reporter molecules is a function of the viral replication rate, a direct correlation occurs between the emitted light rate and the viral replication rate. The use in the method of the invention of a Photon Detector, which assignates an x,y-coordinate and time point for each detected photon, makes possible the correlation between the emitted light rate and the position of the emission source, that is the single cell from which light is emitted. Consequently, the method of the invention, using a bioluminescent reporter gene system, allows the detection and the measurement of the rate of viral replication in a single cell by detecting and measuring the rate of emitted light, particularly of emitted photons, and monitoring of the viral replication kinetics.

Particularly, the method of the invention is useful for quantitative measurement at the single cell level in situ of retroviral gene expression following viral genome integration.

It is understood that the method of the invention can be applied to any virus or retrovirus, as well as to any bioluminescent reporter molecule. Therefore, it constitutes an appropriate tool for quantitative studies on a wide variety of virus-host cell interactions. Particularly, it enables to follow quantitative dynamics of viral gene expression in the virus' living natural target cells or tissues.

The method of the invention harbors enormous potential as a screening assay primarily for identification of host cell specific factors involved in viral replication, and the identification of new antiviral drugs.

The single-cell based virus assay of the invention is not at all limited to cell type. Importantly, it functions equally well in primary preparations of natural target cells, or in clonal cells. This provides the major advantage wherein natural target cells may be targeted either using in vitro or in situ infection paradigms. For example, the method of the invention when applied for single-cell based HIV assay may be used on blood samples isolated from real clinical case studies and can be used for monitoring HIV replication in single cells from said patients.

### DESCRIPTION OF THE INVENTION

The invention relates to a method for the quantitative real-time detection and/or measurement at the single living cell level of viral replication, wherein said method comprises the steps of:
a) providing a recombinant construct, or a vector containing said recombinant construct, comprising at least a part of a viral genome and a bioluminescent reporter gene, wherein the expression of said bioluminescent reporter gene is controlled by a viral promoter comprised in said part of a viral genome;
b) infecting, transfecting or transducing a living cell in vitro with said construct or vector containing said recombinant construct;
c) optionally complementing the system with viral elements necessary for viral replication;
d) culturing said infected, transfected or transduced living cell, optionally complemented during a time sufficient to assume that the replication has at least started;
e) detecting, measuring and optionally cumulating, at the single living cell level, the level of the product expressed by said reporter gene in said infected, transfected or transduced living cell by means of a highly sensitive digital light detection imaging device, which assignates an x,y-coordinate and time point for each detected photon;
   wherein the measured expression level of said product is indicative at the single living cell level of viral replication.

Therefore, the method of the invention allows monitoring viral replication kinetics.

The method of the invention can be used with any virus infected cell culture system already known or developed.

The virus of which the replication can be detected and/or measured by applying the method of the invention can be any virus. The virus can be any DNA virus, RNA virus, mammalian virus, in particular human virus, or plant virus.

The method of the invention preferentially applies, but is not limited to, viruses such as HIV, particularly HIV-1, SIV, and related retroviruses; Influenza virus, Hepatitis viruses, particularly Hepatitis B, Hepatitis C viruses.

According to the invention a preferred retrovirus is HIV-1.

The system can be applied to all HIV-1 strains or isolates independently of lade and tropism, and also to fully replicative viruses.

According to the invention, “viral replication” covers the viral replication of DNA or RNA viruses, and “viral genome” covers the genome of DNA or RNA viruses.

According to this, the cell culture system used in the method of the invention is adapted to each virus the replication of which is to be studied according to the method of the invention. One skilled in the art knows very well and has no difficulties to adapt the cell culture conditions to the particular virus the replication of which is to be studied.

The living cells to which the method applies can be any living cell. It is preferentially a cell that constitutes a natural target for the virus which infection and/or replication has to be quantitatively tracked with the method of the invention. The living cell is more preferentially a primary cell. It can also be a cell from a cell line.

In the case of HIV-1 virus, the living cells are preferentially monocyte derived macrophages and CD4 T lymphocytes from peripheral blood.

According to the invention, “the recombinant construct, or the vector containing said recombinant construct, comprising at least a part of a viral genome and a bioluminescent reporter gene”, means that the recombinant construct or the vector containing said recombinant construct, can contain a non defective complete virus' genome in which the bioluminescent reporter gene has been introduced or a partially deleted virus' genome in which the bioluminescent reporter gene has been introduced, with the proviso that the bioluminescent reporter gene is introduced in the non defective complete virus' genome or in the partially deleted virus' genome at a location non essential for the viral replication, and that the partially deleted virus' genome is deleted at a location non-essential for the viral replication.

Optionally, the recombinant construct or the vector containing said recombinant construct can be complemented by cotransfection of a gene coding for the expression product of the deleted gene.

Advantageously according to the invention, the recombinant construct comprising at least a part of a viral genome and a bioluminescent reporter gene, which is used in step a) of the method of the invention, is such that at least one gene, that is non-essential for the viral replication, is replaced by the bioluminescent reporter gene using conventional recombinant technology.

According to the invention a preferred recombinant construct contains the whole HIV-1 viral genome, except for the nef gene which has been replaced by the bioluminescent reporter gene.

Optionally, some other genes of the viral genome may have been modified, for instance a frame shift can be introduced in the gene coding for the envelope so that the resulting deficient virus can infect the target cell only when an efficient gene encoding for an envelope is co-transfected.

More preferably, the recombinant construct consists of the proviral pNL-Luc-E⁻R⁺ described in Connor et al. (Virology (1995) 206, 935-944) or is derived from said vector.

In the case of the hepatitis C virus, a preferred recombinant construct consists of the plasmid pFK-Luc-JFH1 which encodes a bicistronic reporter construct of the full-length JFH1 genome (Wakita et al. (2005) Nat. Med. 11: 791-6). In this recombinant construct, the HCV polyprotein-coding region is located in the second cistron and the first cistron contains the firefly luciferase reporter gene fused to the JFH1-derived 5′-UTR promoter and the coding region of the 16 amino-terminal residues of core.

According to the invention, any bioluminescent reporter gene can be used in the method, preferably the luciferase gene. Luciferases are enzymes that emit light in the presence of oxygen and a substrate (luciferin) and which have been used for real-time, low-light imaging of gene expression in cell cultures, individual cells, whole organisms, and transgenic organisms. Such luciferin-luciferase systems include, among others, the bacterial lux genes of terrestrial Photorhabdus luminescens and marine Vibrio harveyi bacteria, as well as eukaryotic luciferase luc and ruc genes from firefly species (Photinus) and the sea panzy (Renilla reniformis), respectively. Modified forms of luciferase, especially spectral and/or lysosome targeted (by Pbt) modified forms of luciferase can also be used in the method of the invention.

Preferably according to the invention the luciferase (firefly or renilla) gene is used.

In a particular embodiment, the recombinant construct further comprises a fluorescent reporter gene. Bioluminescent labeling will indicate functional viral gene expression, whereas fluorescence will indicate the sub-cellular compartmentalization of infective virus particles.

A preferred digital light detection imaging device is the customized microscope imaging system capable of detecting very low level light emission that has been described recently (Rogers et al. (2005) Eur J Neurosci 21:597-610). The system comprises a sensitive photon detection system (Imaging Photon Detector IPD) able to discriminate single photon events, assigning an x,y-coordinate and time point to each detected photon (Hooper et al. (1990) J. Biolumin. Chemilumin. 5:123-30).

According to the invention any known and described methods of infecting, transfecting, transducing a living cell, complementing the system with viral elements necessary for viral replication and culturing infected cells can be used in the method.

In one particular embodiment of the invention, the method can be coupled with fluorescent markers that allow consideration of cellular heterogeneity and quantification of viral replication in specific cell subsets of primary and organotypic tissue preparations.

A specific application could be the study of HIV replication and the activation of viral expression in viral cell reservoirs (i.e., resting CD4 T cells or monocytes/macrophages during antiviral therapy) after transduction/infection with a reporter virus.

The invention also relates to the use of the previously described method for the quantitative real-time measurement of the integration and/or transcription of a part of a retroviral genome in the genome of a living cell, wherein the expression level of said product is indicative of the integration and/or transcription level of said part of a retroviral genome in the genome of the infected, transfected or transduced living cell.

The invention also relates to the use of the previously described method for screening a molecule capable of modulating, preferably inhibiting, viral replication in a living cell, wherein a modulation, preferably a decrease, of the expression level of said bioluminescent reporter gene is indicative of a molecule capable of modulating, preferably inhibiting, the viral replication.

The invention also relates to the use of the previously described method for screening a molecule capable of modulating, preferably inhibiting, the integration and/or transcription of a part of a viral genome in the genome of living cell, wherein a modulation, preferably a decrease, of the expression level of said bioluminescent reporter gene is indicative of a molecule capable of modulating, preferably inhibiting, the integration and/or transcription of a part of a viral genome in the genome of a cell.

The invention also relates to a kit designed to perform any one of the previously described methods comprising:
a) a recombinant construct, or vector containing said recombinant construct, comprising at least a part of a viral genome and a bioluminescent reporter gene, wherein the expression of said bioluminescent reporter gene is controlled by a viral promoter comprised in said part of a viral genome;
b) optionally viral elements necessary for viral replication for complementing the system;
c) reagents for the quantitative measurement of the expression level of said bioluminescent reporter gene by means of a highly sensitive digital light detection imaging device, which assignates an x,y-coordinate and time point for each detected photon in a living cell infected, transfected or transduced with said recombinant construct, or vector containing said recombinant construct;
d) optionally, a standard curve or reagents to draw a standard curve.
   In another embodiment of a kit according to the invention, the recombinant construct, or the vector containing said recombinant construct, further comprises a fluorescent reporter gene.

### BRIEF DESCRIPTION OF THE DRAWINGS

FIG. 1. Detection of HIV-1 infection in intact biologically relevant primary cells.

(a) MDM cells were infected with the NL4.3-luc vector pseudotyped with the pantropic VSV-G protein. After 5 days of infection the luciferase activity in the living cells was analyzed. The bioluminescent image shows photons accumulated during 90 seconds.

(b) Activated CD4 T cells were infected with the VSV-G NL4.3-luc pseudotype. Photon emission of the cells three days after infection was integrated during 300 seconds.

(c) The bioluminescence of MDM cells infected with envelope-defective NL4.3-luc particles was also evaluated (integration time=300 seconds).

(d) Infection of MDM cells with R5 tropic BaL pseudotyped HIV-1 particles. The bioluminescent signal was accumulated during 90 seconds. (a-d) The images shown are representative of at least 10 different experiments. The bioluminescent emission (left) is color-coded (photons/pixel, scale at the bottom of the images). The corresponding bright field images are shown in the right panels. Images were obtained employing x10 (MDM) or x25 (CD4 T cells) objectives.

FIG. 2. Quantitative measurement of viral replication by bioluminescence emission in the infected cells. MDM cells were incubated with different amounts of the integrase inhibitor L-731,988 prior to infection with the VSV-G NL4.3-luc pseudotype.

(a) Luciferase activity in lysates. The results, expressed in light units, are given as the mean for three independent experiments. Error bars representing standard deviation are shown in each case.

(b) IPD quantification of overall photon emission per second, per observation field of intact cells. The values are given as the mean±s.d. for n=5 fields.

(c) Bioluminescence imaging of the infected living MDM cells (integration=90 seconds). The number of positive cells per field for each condition (mean±s.d.) is indicated above the images. Each panel is representative of n=5 different fields.

(d) Quantification of photon emission per second in single intact MDM cells (n=25 cells/condition). The boundaries of the boxes closest to zero indicate the 25th percentiles, the lines within the box mark the medians (solid) and the means (long dash), and the boundaries of the boxes farthest from zero indicate the 75th percentiles. Whiskers above and below the boxes indicate the 90th and 10th percentiles. All the outlying points are plotted. Comparisons among data sets were performed by independent sample t-test. All the statistical calculations and graphs were done with Sigma Plot software (Science Products). Images were obtained employing an x10 objective.

FIG. 3. Real-Time imaging and quantification of viral gene expression after stimulation of infected primary cells.

(a-c) Untreated CD4 T cells were infected with the VSV-G NL4.3-luc pseudotype. Three days after infection, PHA (1 μg/ml) was added to the culture and bioluminescence was analyzed over time.

(a) Bioluminescence images (photons accumulated during 300 seconds), superimposed with the brightfield images, of untreated (left) or control CD4 T cells, activated before addition of virus (right), three days after infection (T=0). Images obtained using x25 objective.

(b) Imaging (x40, objective) of cells from the untreated CD4 T cell population at different times after PHA stimulation. One cell (signaled with a white circle) negative for bioluminescence at time 0 (1) and one cell emitting photons before PHA addition (2) are shown.

(c) Changes in photon counts (accumulation during 300 seconds) in the cells shown in panel B (1, black; 2, gray) plotted as a function of time after addition of PHA.

(d) MDM cells were infected with the NL4.3-luc vector pseudotyped with the VSV-G protein. After 5 days of infection PMA was added to the culture (final concentration 30 ng/ml) and the time course of the luciferase activity in the living cells was quantified (gray). As a control the luciferase activity of infected MDM cells was analyzed in the absence of PMA stimulation (black). Photon emission per cell (in 90 seconds) is plotted over time for one representative cell from each population. An x25 objective was used to monitor the cells.

### MATERIALS AND METHODS

Production of Viral Particles Bearing the Luciferase Gene

pNL-Luc-E−R+ is a proviral HIV-1 (NL4.3 strain) plasmid that carries a firefly luciferase reporter gene (replacing the viral gene nef and utilizing the natural Nef translation start site). In addition, this plasmid contains a frameshift close to the viral env gene that avoids expression of the HIV-1 envelope protein. This construct is thoroughly described in (Connor et al. (1995) Virology 206:935-44). Single-cycle competent HIV-1 particles bearing the luciferase reporter gene were produced by transiently co-transfecting (SuperFect, Qiagen) 5×10⁶ 293T cells with the proviral pNL-Luc-E−R+ (7.5 μg) and the HIV-1BaL-Env expression vector (15 μg), coding for the envelope protein of the HIV-1 BaL strain, or the VSV-G expression vector (7.5 μg), coding for the G protein from vesicular stomatitis virus, which has a ubiquitous (pantropic) cellular receptor (Aiken (1997) J. Virol. 71:5871-7) and ensures a high efficiency of infection. Culture supernatants were harvested 48 h after transfection of the cells, filtered and stored at −80° C. Production of viral particles was evaluated by ELISA quantification (Beckam Coulter, Paris, France) of the viral p24 in the filtered supernatants. Supernatants of cells transfected only with pNL-Luc-E−R+ were used as controls.

Primary Cell Cultures of Monocytes Derived Macrophages and CD4 Cells

Human peripheral blood mononuclear cells (PBMC) were isolated from buffy coats of healthy seronegative donors (Centre de Transfusion Sanguine Ile-de-France, Rungis or Hôpital de la Pitié-Salpêtriére, Paris, France) by Ficoll-Hypaque (Pharmacia Biotech) density gradient centrifugation. Monocytes were isolated by adherence to plastic and thereafter allowed to differentiate into macrophages for 10 days in hydrophobic Teflon bags (Lumox; Dominique Dutscher) in RPMI 1640 medium supplemented with 200 mM L-glutamine, 100 U of penicillin, 100 μg of streptomycin, 10 mM HEPES, 10 mM sodium pyruvate, 50 μM 2-ME, 1% MEM vitamins, and 1% nonessential amino acids (MDM medium) in presence of 15% of human AB serum as previously described (Perez Bercoff et al. (2003) J. Virol. 77:4081-94). Monocyte derived macrophages (MDM) were then harvested, washed and resuspended in MDM medium containing 10% heat-inactivated fetal calf serum (FCS) for experiments. CD4 cells were purified (>99% as estimated by flow cytometry analysis) from PBMC by positive selection employing antibody-coated immunomagnetic beads (Miltenyi Biotech, France) as specified by the manufacturer. CD4 T cells were cultured in RPMI 1640 medium supplemented with 200 mM L-glutamine, 100 U of penicillin, 100 μg of streptomycin in the presence of interleukin-2 (IL2) (Chiron, France) (100 Ul/ml). When needed, CD4 T cells were activated for three days with phytohemagglutinin (PHA) (1 μg/ml) before infection.

Infections

Viral inputs (140 or 450 ng p24 of the HIV-1 VSV-G or Bal-Env pseudotypes respectively) and incubation times (macrophages and lymphocytes were analysed for steady stated single-cell bioluminescence 5 and 3 days after infection) were chosen on the basis of their yield of a maximal signal in parallel experiments using conventional cell lysate measurements in a luminometer. Different cell concentrations were assayed to obtain optimal cell density for imaging analysis. Finally 10⁵ MDM or 10⁶ CD4 T cells were challenged with HIV-1 particles bearing the luciferase reporter gene, plated on the centre of glass-bottomed dishes (MatTek corp, MA, USA) and incubated at 37° C. for two hours. The cells were then washed extensively and finally 1 ml of complete appropriate culture medium was added to each plate for culture.

Optionally, in order to enhance infection, viral challenge was carried out with a spinoculation protocol (1 hour centrifugation at room temperature at 1500×g) (O'Doherty et al. (2000) J. Virol. 74:10074-80).

Photon Quantification in Cell Lysates

At the indicated times postinfection 10⁵ cells were harvested and lysed with 100 μl of luciferase lysis buffer (Promega). T he luciferase activity was quantified in 20 μl of each lysate with the Promega Luciferase Assay System and a luminometer LUMAT LB9501 (Berthold Technologies).

Description of the Photo Counting System and Photon Quantification in Living Cells

Cell luminescence was measured using a wide-field microscope system customized for bioluminescence (ScienceWares Inc., MA, USA), as previously described (Rogers et al. (2005) Eur. J. Neurosci. 21:597-610). The system includes a fully automated inverted microscope (200M, Carl Zeiss, Germany) housed in a light-tight dark box. Low level light emission was collected using an Imaging Photon Detector (IPD 3, Photek Ltd. East Sussex, UK) connected to the baseport of the microscope. In this configuration the background signal of the IPD camera was extremely low (<1 photon s in a 256·256 pixel region). The system is fully controlled by the data acquisition software, which also converts single photon events into an image that can be superimposed with bright-field images obtained with a CCD camera (Coolsnap HQ, Roper Scientific). Observations were made with Plan-Neofluar 10× (air, NA=0.5), 25× (oil, NA=0.8) and 40× (oil, NA=1.3) objectives (Carl Zeiss, Germany).

Conditions for Steady-State and Dynamic Analyses

At the desired post-challenge times the medium in the plates was replaced by PBS+5% FCS (for steady-state analysis) or complete phenol red free medium (for dynamic analysis). To facilitate analysis, the non-adherent CD4 T cells were transferred to new MatTek plates coated with poly-L-lysine (15 minutes incubation with 1 mg/ml poly-L-lysine followed by washing and drying of the poly-L-lysine-coated solid phase) or simply left to settle down for 30 minutes before analysis. Endotoxin free beetle D-luciferin (Promega) was added to the plates that were subsequently placed on the inverted microscope. Different luciferin concentrations were assayed in order to find those providing detectable and durable bioluminescence signals without affecting cell viability. Luciferin concentrations ranging from 0.001 to 1 mM provided detectable enough signal to allow analysis. Optimal concentrations providing maximal signal depended on cell type (1 mM for macrophages and 0.1 mM for CD4 T cells).

The steady state analyses were conducted at room temperature (optimal for luciferase activity). Alternatively, the glass-bottomed dishes were placed in a temperature-controlled (37° C.) chamber for the dynamic studies, and D-luciferin was replenished every 6-8 hours. Optimal culture conditions can be further achieved with a temperature controlled chamber that also supplies CO₂.

Detection of Photon Emission and Analysis

To determine optimal photon integration times for the different cell systems, we conducted setting up experiments in which different fields of infected cells were visualized using the bioluminescence system by accumulating photon counts during extended periods of time lasting up to 5-6 h. Integration times leading to unequivocal identification of positively infected photon emitting cells were chosen (90 seconds for macrophages and 300 seconds for CD4 T cells).

For the quantification of photon emission in single cells, the cell outline in the bright-field image was used to define the region of interest, and only the photons detected in that area were taken into account.

Flow-Cytometry Analysis of Activation Markers

CD4+ T cell populations were checked for membrane expression of activation markers CD25 and CD69 by three-color flow cytometry analysis. The cells were incubated for 30 minutes at room temperature with a mixture of CD69-PE (BD Bioscience), CD25-FITC and CD4-ECD (Beckman Coulter). After staining, the cells were washed in PBS, adjusted to 500 μL in PBS+5% FCS and analyzed on a Cytomics FC500 flow cytometer (Beckman Coulter). 20,000 living cell events were accumulated on the lymphocyte gate that was set up using both forward and right angle scatters.

### EXAMPLES

### Example 1

### Detection of HIV-1 Infection in Living Primary Target Cells

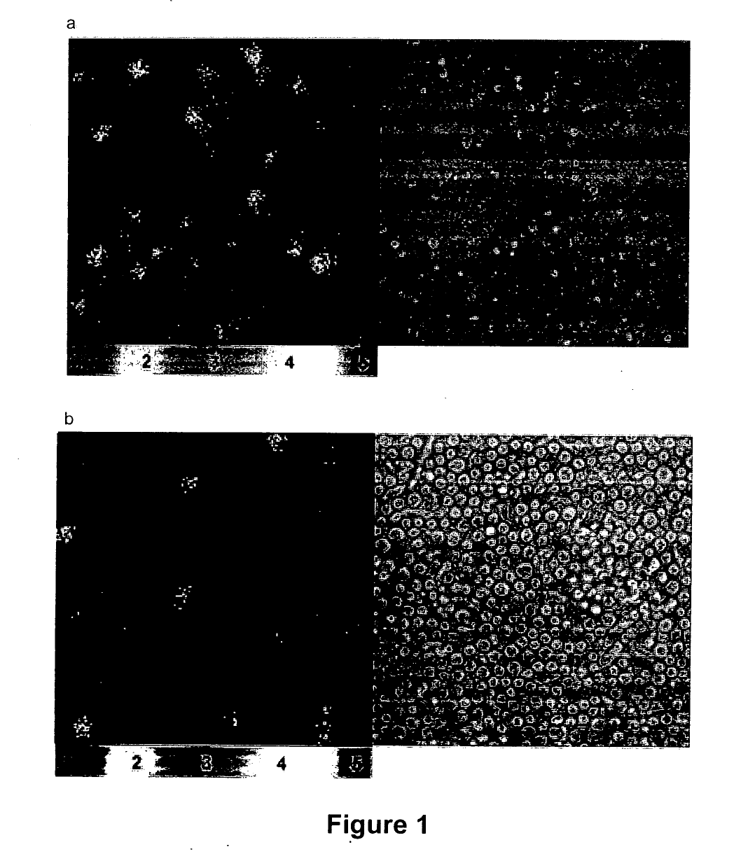

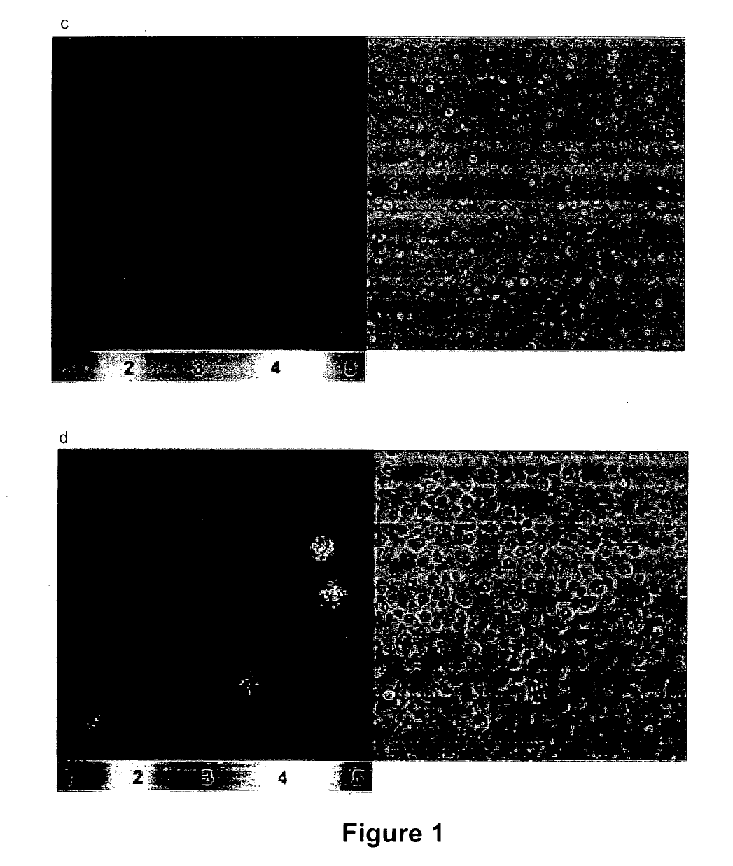

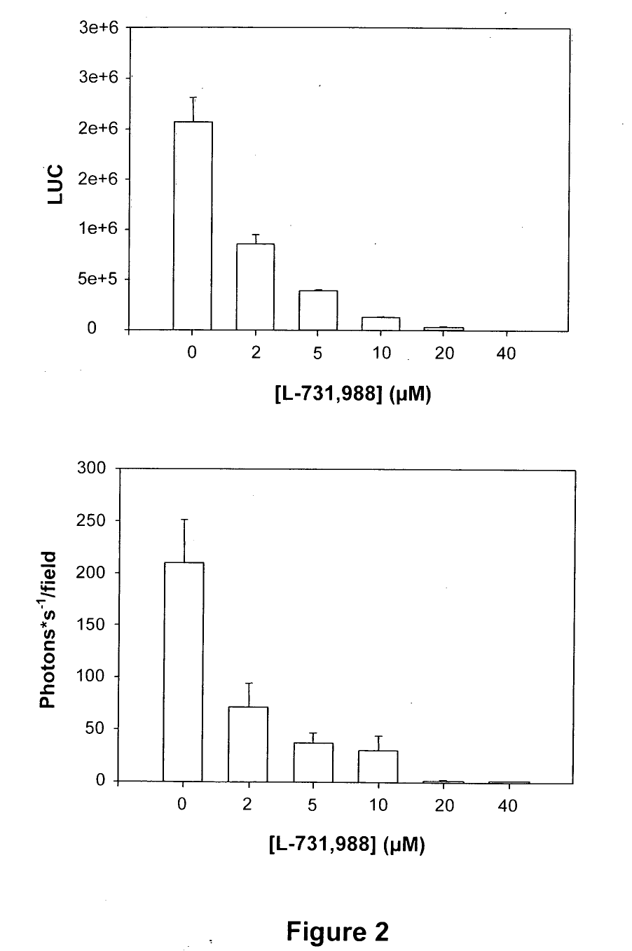

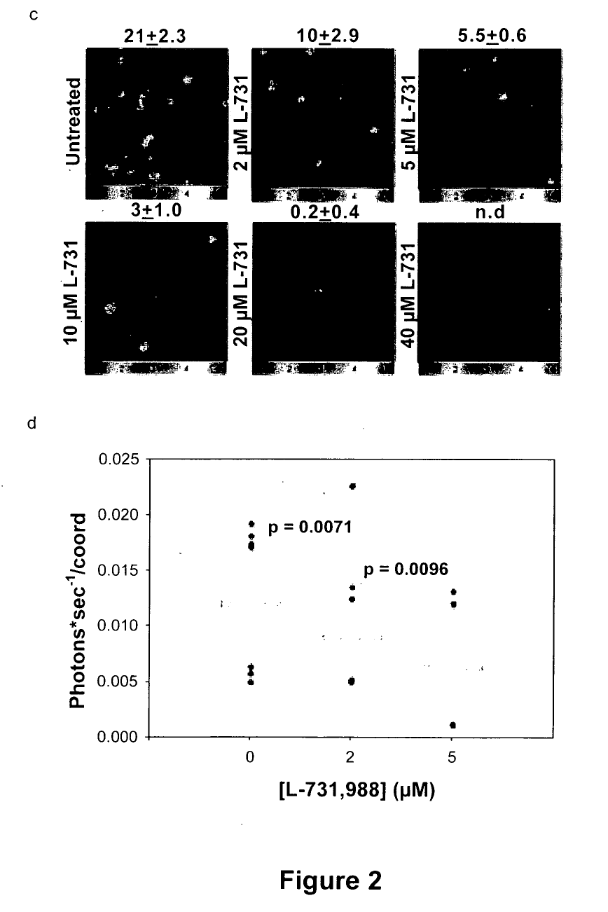

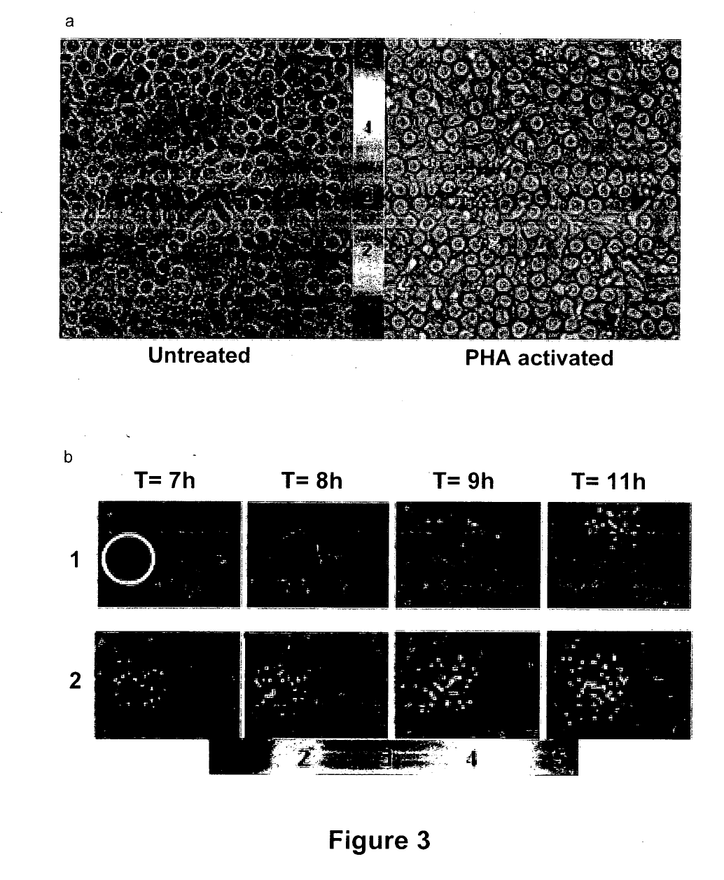

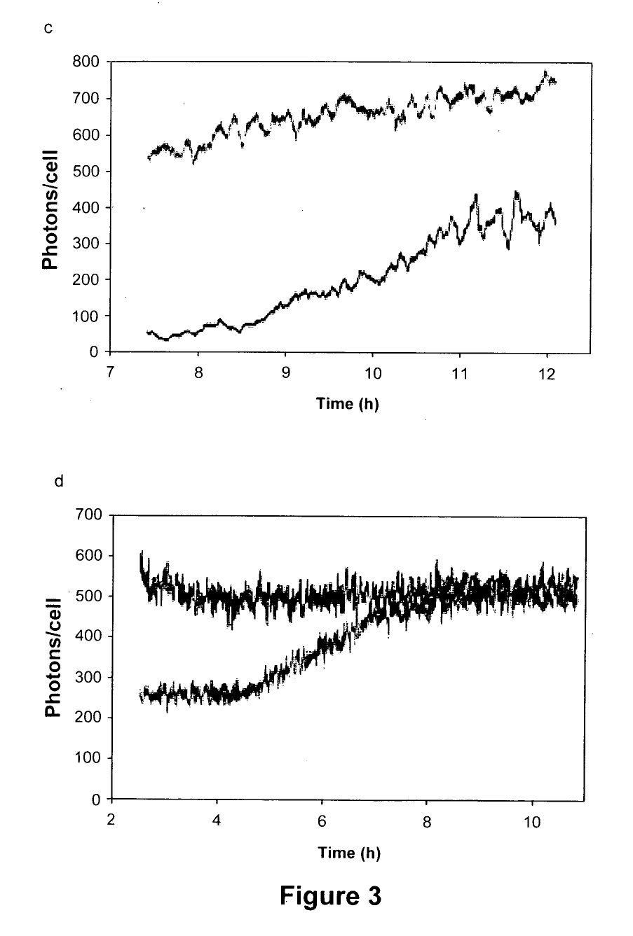

We tested the performance of the bioluminescence system in single round infections of two major natural HIV-1 target cells: human monocyte derived macrophages (MDM) (FIG. 1a) and activated CD4 T lymphocytes from peripheral blood (FIG. 1b). Target cells were infected with NL4-3-luc HIV-1 particles, engineered such that part of the viral nef gene is replaced by the luciferase reporter gene (Connor et al. (1995) Virology 206:935-44), pseudotyped with the Vesicular Stomatitis Virus protein G (VSV-G) that uses a membrane phospholipid as a receptor for entry and yields a high efficiency of infection compared with HIV-1 envelopes (Aiken (1997) J. Virol. 71:5871-7). Cells were checked for bioluminescence emission 5 and 3 days after infection respectively, times that yielded for each cell system maximal luciferase activity in cell lysates (not shown). Emission of bioluminescence was readily detected in the infected cells upon addition of luciferin (FIG. 1a,b). As expected, no bioluminescent signal was observed when envelope defective viral particles where employed for the infection of MDM (FIG. 1c) or of CD4 T cells (not shown). In addition, we infected the macrophages with NL4.3-luc particles pseudotyped with an R5 tropic HIV-1 envelope (BaL). Although, as expected, a lower number of infected cells were detected than with VSV-G pseudotyped virus, significant numbers of infected cells were observed, and easily distinguished from negative cells (FIG. 1d). These results provided the proof-in-principle that our imaging set-up facilitated an efficient means for detection of individual cells expressing luciferase following infection with HIV-1.

### Example 2

### Quantifying HIV-1 Infection at the Single Cell Level

In order to establish whether our method for detection of bioluminescent signals could be used as a way to quantify single-cell infection levels, we examined the effect of the diketo acid L-731,988, an integrase inhibitor which prevents HIV-1 integration by blocking strand transfer (Hazuda et al. (2000) Science 287:646-50). MDM cells were incubated in the presence of increasing concentrations of L-731,988 prior to, and during infection with the HIV-1 VSV-G pseudotype. Five days after infection we measured infection levels both using a luminometer in cell-lysates (Connor et al. (1995) Virology 206:935-44), and in intact single cells using our bioluminescence imaging system. As expected, the luciferase activity in cell lysates treated with the integrase inhibitor was reduced in a dose-dependent manner indicative of inhibition of HIV-1 replication in MDM cells (FIG. 2a). In parallel, the single-cell bioluminescence imaging assay showed that the overall averaged bioluminescence intensity also decreased (FIG. 2b) in part due to a reduction in the number of cells emitting bioluminescence (FIG. 2c). Thus, results from quantitative analysis of cell lysates using a luminometer, and intact living cultured cells using our single-cell photon imaging system were concomitant; suggesting strongly the quantitative utility of the latter methodology. Accordingly, we analysed the photon emission from individual cells and found further that the averaged decrease in the bioluminescence signal was, in part, also due to a significant reduction in the number of photons emitted per individual cell (FIG. 2d). That the integrase inhibitor L-731,988 caused a reduction in both the number of bioluminescent cells detected, and the measured intensity of bioluminescence produced by individual cells is wholly consistent with its expected action to cause a dose-dependent shut-down of HIV-1 replication. We concluded, therefore, that HIV-1 dependent bioluminescence signals provide a valid and useful means to quantify HIV-1 infection even at the single cell level.

### Example 3

### Monitoring HIV-1 Gene Expression Dynamics in Single Living Primary Cells

In situ, under normal conditions the vast majority of CD4 T lymphocytes in the peripheral blood and lymphoid tissues are in a resting (non-activated) state, and represent a latent reservoir for HIV-1 (Pierson et al. (2000) Annu Rev Immunol 18:665-708). Along these lines, activated CD4 T lymphocytes support rapid HIV-1 replication, whereas resting CD4 T cells are incapable of sustaining infection due to several blocks during early steps of viral replication (Chiu et al. (2005) Nature 435:108-14, Ganesh et al. (2003) Nature 426:853-7). Indeed, in resting CD4 T cells HIV-1 DNA is predominantly unintegrated, but cellular activation (eg: mitogenic stimulation) can overcome this, allowing virus integration and replication (Pierson et al. (2002) J. Virol. 76:8518-31). Based upon our results showing that bioluminescence signals in single cells are a direct indicator of viral gene expression efficiency therein, we next used this method to investigate reactivation of HIV-1 from latency in individual infected cells.

Total CD4 T cells were isolated from peripheral blood mononuclear cells. After positive CD4 selection the percentage of resting cells was 90%, as reflected by the very low proportion of cells expressing the T cell activation markers CD25 and CD69. Three days later, at the time of infection, the proportion of resting cells remained virtually unchanged (89%). Evidently, cells monitored using single cell level bioluminescence imaging allowed the experiments to be performed on heterogeneous cell mixtures, without need for further purification among the different cell subpopulations. Cells were monitored three days after infection inasmuch as this is the time required to detect complete reverse transcription in resting CD4 T cells (Pierson et al. (2002) J. Virol. 76:8518-31); and is longer than the time necessary to monitor one round of viral replication in activated cells. As shown in FIG. 3a (left) in non-activated CD4 T cells infected with the VSV-G pseudotyped HIV-luc particles, we occasionally observed HIV-1 bioluminescence under resting conditions, presumably due to the infection of some of the already activated T cells (thereafter referred as pre-activated CD4 T cells). This expected result provided a convenient internal control for the functional assay. By contrast, parallel observations of infected cells treated, before infection, with phytohemagglutinin (PHA) during three days (to induce a global activation of CD4 T cells (>98%, not shown) revealed abundant numbers of positive lymphocytes (FIG. 3a, right). Based on this result we next examined the kinetics of the PHA driven viral gene expression rescue. PHA was added to the infected non-activated cell cultures in order to render the resting cells fully permissive to viral replication, and cells were monitored over time. Under these conditions, as soon as 8 hours after mitogen stimulation, photon emission began to be detected in formerly negative cells (FIG. 3b). This emission increased significantly during 4 hours, reaching a stable plateau level (black line (1), FIG. 3c) comparable to levels detected in both infected, pre-activated cells present in the same culture (gray line, FIG. 3b,c), and control CD4 T cells activated with PHA before infection (not shown). The fast PHA triggered response was not unexpected. Signaling and nuclear translocation of factors critical for HIV-1 replication occurs minutes following stimulation of T cells (Kinoshita et al. (1997) Immunity 6:235-44, Timmerman et al. (1996) Nature 383:837-40), and changes in gene expression after activation of naive CD4 T cells have been reported to take place as soon as 15 minutes after stimulation (Hooper et al. (1990) J. Biolumin. Chemilumin. 5:123-30). Thus, the changes needed to overcome the replication blocks in resting cells can occur rapidly after mitogenic stimulation, making infected cells permissive to HIV replication. Actually, it has been reported that less than 6 hours are necessary to reactivate HIV-1 from latency in both T cell and monocyte/macrophage lines (Kutsch et al. (2002) J. Virol. 76:8776-86).

Finally, aiming to further demonstrate the utility of our method as a means to follow dynamic variations in HIV-1 gene expression, we next measured the effect of phorbol 12-myristate 13-acetate (PMA) stimulation on infected primary macrophages. MDM infected with the HIV VSV-G pseudotype were stimulated with PMA, which is expected to rapidly enhance HIV-1 transcription (Kaufman et al. (1987) Mol. Cell. Biol. 7:3759-66) and the photon emission of single cells was monitored during several hours. As shown in FIG. 3d, the bioluminescence signal of these cells began to increase just 4 hours after PMA stimulation. This comparatively rapid change in the photon emission was clearly associated to the PMA stimulation of transcription of the integrated proviruses as no increase was observed in non-stimulated infected macrophages (FIG. 3d).

1. A method for the quantitative real-time detection and/or measurement at the single living cell level of viral replication, wherein said method comprises the steps of:
a) providing a recombinant construct, or a vector containing said recombinant construct, comprising at least a part of a viral genome and a bioluminescent reporter gene, wherein the expression of said bioluminescent reporter gene is controlled by a viral promoter comprised in said part of a viral genome;
b) infecting, transfecting or transducing a living cell in vitro with said construct or vector containing said recombinant construct;
c) optionally complementing the system with viral elements necessary for viral replication;
d) culturing said infected, transfected or transduced living cell, optionally complemented during a time sufficient to assume that the replication has at least started;
e) detecting, measuring and optionally cumulating, at the single living cell level, the level of the product expressed by said bioluminescent reporter gene in said infected, transfected or transduced living cell by means of a digital light detection imaging device, which assigns an x,y-coordinate and time point for each detected photon;
wherein the measured expression level of said product is indicative at the single living cell level of viral replication.

2. The method according to claim 1, wherein the virus of which the replication can be detected and/or measured is any virus, particularly any DNA virus, RNA virus, mammalian virus, or plant virus.

3. The method according to any one of claims 1 and 2, wherein the virus is selected from HIV, SIV, Influenza virus, and Hepatitis viruses.

4. The method according to claim 3, wherein the HIV virus is HIV-1.

5. The method according to claim 1, wherein the living cell is a cell that constitutes a natural target for the virus.

6. The method according to claim 5, wherein the cell is a primary cell.

7. The method according to claim 5, wherein the cell is a cell from a cell line.

8. The method according to claim 1, wherein the recombinant construct contains the whole HIV-1 viral genome, except for the nef gene, which has been replaced by the bioluminescent reporter gene.

9. The method according to claim 1, wherein the recombinant construct consists of the proviral pNL-Luc-E−R⁺ or is derived from said vector.

10. The method according to claim 1, wherein the bioluminescent reporter gene is chosen from the bacterial lux genes of terrestrial Photorhabdus luminescens and marine Vibrio harveyi bacteria and eukaryotic luciferase luc and ruc genes from firefly species (Photinus) and the sea panzy (Renilla reniformis).

11. The method according to claim 10, wherein the bioluminescent reporter gene is the luciferase (firefly or renilla) gene.

12. The method according to claim 1, wherein the recombinant construct, or the vector containing said recombinant construct, further comprises a fluorescent reporter gene.

13. The method according to claim 12, wherein viral replication kinetics and sub-cellular compartmentalization of infective virus particles are monitored simultaneously.

14. The method according to claim 1, wherein the digital light detection imaging device is a customized microscope imaging system equipped with an Imaging Photon Detector capable of detecting very low level light emission.

15. The method according to claim 14, wherein the system comprises a sensitive photon detection system able to discriminate single photon events, assigning an x,y-coordinate and time point to each detected photon.

16. The method according to claim 1, wherein the method quantitates real-time measurement of the integration and/or transcription of a part of a retroviral genome in the genome of a living cell and wherein the expression level of said product is indicative of the integration and/or transcription level of said part of a retroviral genome in the genome of the infected, transfected or transduced living cell.

17. The method according to claim 1, wherein the method screens a molecule capable of modulating, preferably inhibiting, viral replication in a living cell and wherein a modulation, preferably a decrease, of the expression level of said bioluminescent reporter gene is indicative of a molecule capable of modulating, preferably inhibiting, the viral replication.

18. The method according to claim 1, wherein the method screens a molecule capable of modulating, preferably inhibiting, the integration and/or transcription of a part of a retroviral genome in the genome of living cell and wherein a modulation, preferably a decrease, of the expression level of said bioluminescent reporter gene is indicative of a molecule capable of modulating, preferably inhibiting, the integration and/or transcription of a part of a retroviral genome in the genome of a cell.

19. A kit for the quantitative real-time measurement at the single living cell level of viral replication comprising:
a) a recombinant construct, or vector containing said recombinant construct, comprising at least a part of a viral genome and a bioluminescent reporter gene, wherein the expression of said bioluminescent reporter gene is controlled by a viral promoter comprised in said part of a viral genome;
b) optionally viral elements necessary for viral replication for complementing the system;
c) reagents for the quantitative measurement of the expression level of said bioluminescent reporter gene by means of a digital light detection imaging device, which assigns an x,y-coordinate and time point for each detected photon in a living cell infected, transfected or transduced with said recombinant construct, or vector containing said recombinant construct;
d) optionally, a standard curve or reagents to draw a standard curve.

20. The kit according to claim 19, for the quantitative real-time measurement of the integration and/or transcription of a part of a retroviral genome in the genome of a cell comprising:
a) a recombinant construct, or vector containing said recombinant construct, comprising at least a part of a viral genome and a bioluminescent reporter gene, wherein the expression of said bioluminescent reporter gene is controlled by a viral promoter comprised in said part of a viral genome;
b) optionally viral elements necessary for viral replication for complementing the system;
c) reagents for the quantitative measurement of the expression level of said bioluminescent reporter gene by means of a digital light detection imaging device, which assigns an x,y-coordinate and time point for each detected photon in a living cell infected, transfected or tranduced with said recombinant construct, or vector containing said recombinant construct;
d) optionally, a standard curve or reagents to draw a standard curve.

21. The kit according to claims 19 or 20, wherein said viral genome is selected from HIV genome, SIV genome, Influenza virus genome, and Hepatitis virus genome.

22. The kit according to claim 19, wherein the bioluminescent reporter gene is selected from luciferase and modified forms of luciferase.

23. The kit according to claim 19, wherein the living cell is selected from a cell that constitutes a natural target for the virus, a primary cell, and a cell from a cell line.

24. The kit according to claim 19, wherein the recombinant construct, or the vector containing said recombinant construct, further comprises a fluorescent reporter gene.

25. The method according to claim 2, wherein the mammalian virus is a human virus.

26. The method according to claim 3, wherein the Hepatitis virus is selected from Hepatitis B virus and Hepatitis C virus.

27. The kit according to claim 21, wherein said HIV genome is an HIV-1 genome.

28. The kit according to claim 21, wherein said Hepatitis virus genome is selected from a Hepatitis B virus genome and a Hepatitis C virus genome.

29. The kit according to claim 22, wherein the luciferase is selected from firefly luciferase and renilla luciferase.

30. The kit according to claim 22, wherein the modified form of luciferase is spectral and/or lysosome targeted (by Pbt) modified forms of luciferase.
